(19) United States
(12) Patent Application Publication
(10) Pub. No.: US 2003/0212030 A1
(43) Pub. Date: Nov. 13, 2003

(54) Novel adenoviral vector for transferring human genes in vivo

(75) Inventor: Wolfgang Christian Poller, Wurzburg (DE)

Correspondence Address: FINNEGAN, HENDERSON, FARABOW, GARRETT & DUNNER, LLP, 1300 I STREET, NW, WASHINGTON, DC, 20005, US

(73) Assignee: Centeon Pharma GmbH.

(21) Appl. No.: 10/424,996

(22) Filed: Apr. 29, 2003

Related U.S. Application Data
(63) Continuation of application No. 08/861,323, filed on May 21, 1997, now abandoned.

(30) Foreign Application Priority Data
May 22, 1996 (DE) 196 20 687.1

(51) Int. Cl. A61K 48/00; C12N 15/861

(52) U.S. Cl. 514/44; 424/93.2; 435/456

(57) ABSTRACT

The invention relates to a novel adenoviral vector for transferring human genes in vivo. The fields to which the invention can be applied are medicine and the pharmaceutical industry.

The invention relates to a novel adenoviral vector for transferring human genes in vivo. The fields to which the invention can be applied are medicine and the pharmaceutical industry.

The medicinal treatment of the serious monogenic hereditary diseases hemophilia A and hemophilia B is based on using intravenous infusion, which normally has to be carried out about 3 times weekly, to replace the coagulation factors, Factor VIII and Factor IX, respectively, which the patient is lacking. If inhibitors are formed against the coagulation factors which have been replaced, and also if factor consumption has increased (for example in the context of surgical interventions being carried out on the patient), it is sometimes necessary to administer factors several times daily. This procedure, which has to be kept up throughout life, is psychologically stressful for the patient. In this respect, it would be desirable if a therapeutic process were available which permitted substantially longer treatment intervals (for example of the order of magnitude of months) . However, this is not possible when exogenously supplied factors are used since these factors only have a very short half-life in the recipient organism

Another aspect of present day hemophilia therapy relates to the safety of the procedure. In the case of factor concentrates derived from a human donor plasma pool, there is in principle the possibility that infectious agents which were previously unknown or cannot be identified with certainty are carried over together with the concentrates, although inactivation procedures which have by now been introduced, and the individual purification steps themselves, inactivate or remove such agents. The use of recombinant human coagulation factors which have been prepared in eukaryotic cell lines reduces any such risk quite substantially, even if it is not possible to absolutely exclude the existence of latent, cryptogenic viruses in the production cell lines. However, it is not possible to achieve an extension of the treatment interval even using the recombinant factors.

The availability of methods for transferring cloned genes in vivo, that is in the intact organism, has in principle opened up the possibility of effecting so-called “somatic gene therapy” of the hemophilias. If it were possible firstly to introduce the human genes for Factor VIII or Factor IX into the patient at high efficiency (preferably into the liver as the physiological site of synthesis) and secondly to ensure their expression over a protracted period by means of one single administration or by means of repeated administrations at relatively long time intervals (of several weeks up to years), this would represent a very substantial advance in the treatment of hemophilia patients, if no relevant side effects were to occur

Since 1990, various gene transfer systems have been developed with the long-term aim of using them for the purpose of somatic gene therapy. One of the most promising systems is that represented by the so-called replication-defective adenoviral vectors, which ensure a very high gene transfer efficiency, such as cannot be achieved by any other presently available system, not only ex vivo but also in vivo in the intact whole organism. However, in its present form, the adenoviral vector system still suffers from two serious deficiencies. On the one hand, the duration of expression in vivo is limited (to a few weeks in most model systems) and, on the other hand, the vector itself triggers immunological reactions in the recipient which not only accelerate elimination of the vector from the target tissue but also give rise in that tissue to immunopathological phenomena. If the problems of the adenoviral system which have been outlined above could be overcome, this would represent a substantial advance on the road to a long-term gene therapy of monogenic, and other, diseases.

The target of the invention is the further development of the adenoviral vector system with the aim of overcoming the key problems which have been mentioned. The object of the invention is to configure the transfer of genes by means of adenoviral vectors into the liver in such a way that a considerably more prolonged transgene stability is achieved as compared with previous methods.

The invention is implemented in accordance with claims 1 to 6, with the subordinate claims being preferred variants.

The center-piece of the invention is the construction of a novel replication-defective adenoviral vector which comprises, as an important element, the complete E3 region of wild-type adenovirus (preferably Serotype 5), where appropriate containing specific alterations to this region with the aim of amplifying the expression of E3 genes (by incorporating strong, constitutively active promoters such as that of cyto-megalovirus), and the use of this vector for gene transfer while at the same time subjecting the recipient organism to a transient anti-CD4 treatment. The anti-CD4 treatment is preferably carried out using suitable monoclonal antibodies against CD4 antigens, which treatment chronologically overlaps the administration of the adenoviral vector for the gene transfer. The important element of the invention is the combination of the E3-positive vector with the anti-CD4 strategy in order to improve hepatic gene transfer.

Examples of suitable monoclonal anti-CD4 antibodies are those which block signal transduction from the CD4 receptor or deplete the target organism of CD4-positive lymphocytes. Appropriate humanized monoclonal antibodies are particularly preferred in each case.

The further development of the adenoviral vector system in accordance with the invention enables the therapy of various diseases to be improved substantially. Use of these vectors (E3-positive, E3-amplified vectors) in combination with transient anti-CD4 treatment for inducing tolerance should enable the treatment of the monogenic hereditary diseases hemophilia A and hemophilia B to be improved.

The invention is clarified below by means of exemplary embodiments.

### EXAMPLE 1

Construction of the Vector and Incorporation of the Gene

A. Incorporation of Human F IX cDNA into an Expression Plasmid

F IX cDNA was prepared from human liver total RNA by means of a reverse transcriptase-coupled polymerase chain reaction (RT-PCR), as follows. The poly A⁺mRNA, which was isolated using RNeasy® (Quiagen Inc.), was purified from the total RNA using oligotex® (Quiagen Inc.) and employed for synthesizing the coding cDNA using AMV RT (Boehringer Mannheim). This was followed by the RT-PCR, for which thermostable AmpliTaq® DNA polymerase (Applied Biosytems Inc.) was used. The resulting DNA was first of all cloned into a PCR-Script® plasmid (Stratagene Inc.) and then sequenced. The F IX cDNA, which was verified by sequencing, was excised from the abovementioned cloning vector and inserted into the expression plasmid pZS2, which is suitable for eukaryotic cells; as the components which are important in this context, plasmid pZS2 contains an adenovirus sequence from the 5′ ITR sequence up to nucleotide position 445, followed by the CMV promoter, a polylinker sequence, termination signals of the bovine growth hormone gene and a unique Xbal restriction site which is used to clone the linearized F IX/pZS2 plasmid into the adenovirus vector RR5 (see below). When this is done, the F IX structural gene is ligated in in such a way that it is under the control of the strong CMV promoter/enhancer.

B. Construction of the E3+ Adenovirus Component

The source of the adenovirus genome which was suitable for the present purpose was the RR5 virus, which contains a deletion in the El region (from nucleotide position 445 to nucleotide position 3333). This deletion makes the virus replication-deficient. However, the complete E3 region of adenovirus type 5(Ad 5) is conserved in RR5. RR5 was multiplied in human embryonic kidney cells strain 293 and was obtained following isolation by means of CsCl density gradient centrifugation and subsequent desalting by column chromatography on Sephadex® (Pharmacia AB) G-25. Viral DNA was isolated from the abovementioned virus preparation and cut with Xba 1 restriction endonuclease, which dissociates the genome into a large 3′-terminal part and a smaller, 445 kb, 5′-terminal part.

The recombinant, linearized F IX/pZS2 plasmid is now ligated to the large Xba1 fragment from RR5 and multiplied in the abovementioned kidney cells after the latter have been transfected with the ligation product using the calcium phosphate method. Finally, the sought-after vector, Ad 5E3⁺ [symbol] E1 F IX, was obtained from single plaques using the known standard plaque purification method and monitoring by means of F IX-specific PCR analysis.

### EXAMPLE 2

Vector Expression in Primary Human Vascular Endothelial Cells (HUVEC)

For monitoring purposes, confluent primary human endo-thelial cell cultures (HUVEC), which were stable in culture for up to 4 weeks, were in each case infected, at a multiplicity of infection (MOI) of 10, with an adenovirus luciferase vector or the Ad5E3⁺F IX vector carrying the factor IX gene. Both F IX:Ag and F IX:c activity, and also luciferase activity, were detect-able, in each case with comparable expression kinetics, over a period of 4 weeks without pathological changes thereby being induced in the endothelial cell mono-layers.

### EXAMPLE 3

Long-term Expression of the E3⁺F IX Vector in the Mouse

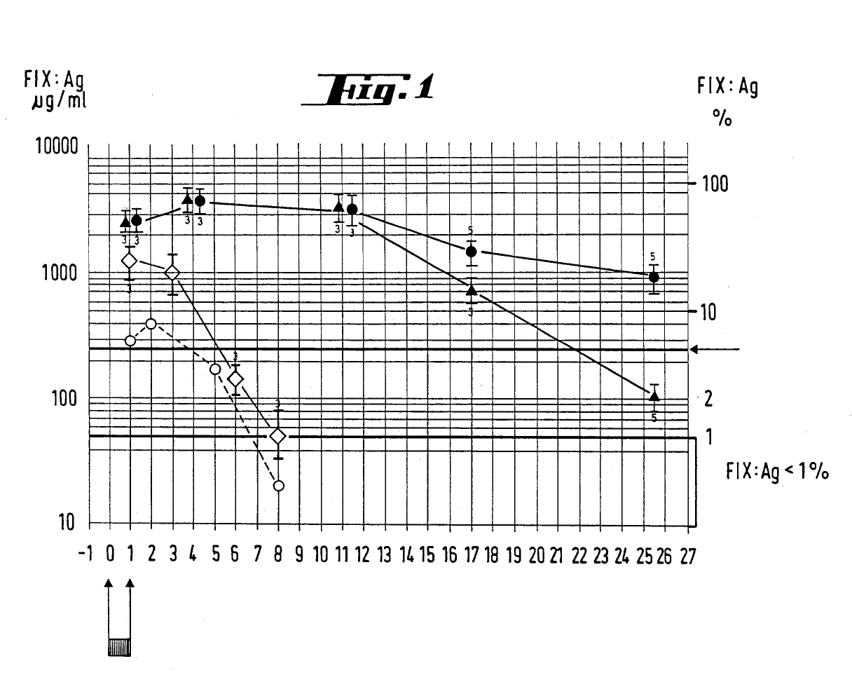

Vector Ad5E3⁺F IX, containing the DNA for human factor IX, was injected intravenously into mice at a dose of 2×10¹⁰ PFU per animal. In all,. 20 mice were treated in this way with 10 of them additionally being given a transient anti-CD4 treatment. Adenoviral vectors recog-nize the liver as their target organ; the expression activity of the F IX transgene in the liver was measured in the plasma of the vector-injected mice by means of determining the recombinant human F IX:Ag in a. a percentage of the value in normal human plasma (100% value at 5,000 μg/ml). The factor IX:Ag concentrations, which were initially <1% (<50 μg/ml) in the untreated mice, increased in some animals to 120% (6,000 μg/ml). All the treated mice had concentrations of F IX:Ag which were in the vicinity of the physiological values for the first three months of the study (FIG. 1).

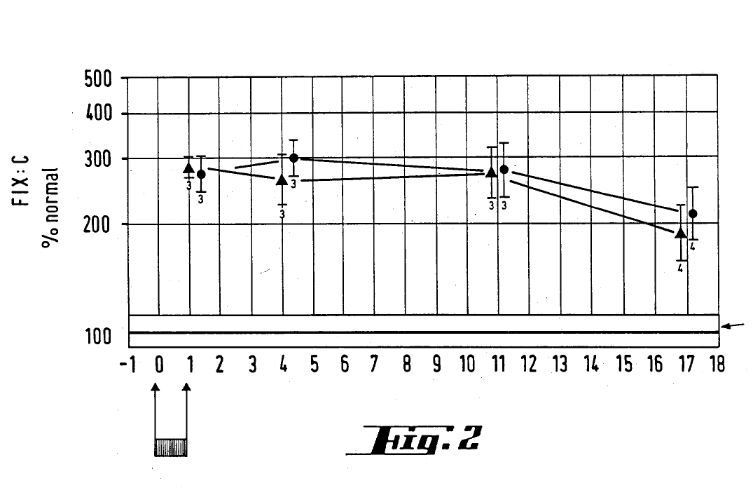

The recombinant F IX was also functionally active, as was demonstrated by a test for total F IX activity (F IX:C) in the mouse plasma using a chromatogenic sub-strate (FIG. 2). The base value for untreated mice was 102±8%. The F IX:C values following transfer of the F IX gene are given as the percentage value of the activity of normal human pool plasma (=100%). After treatment with vector Ad5E3 F IX, the values of F IX:C in mouse plasma rapidly rose to values of up to 320% in some animals. At the same time, the time course curves are similar to those obtained with recombinant F IX:Ag.

The human F IX:Ag reached values of about 60 (3000 μg/ml) after only a week post-injection. In both the immunocompetent and the anti-CD4-treated animals, the human F IX:Ag values were at a similar level, fairly uniformly in the vicinity of the physiological values, for up to 10 weeks after the gene transfer and remained clearly above the therapeutic threshold value of 5 (250 mg/ml) for a total 4 months.

The F IX expression values which were obtained using the E3-deleted vector (Av1H9B), which was employed in an earlier study (Smith et al., “Adenovirus-mediated expression of therapeutic plasma levels of human factor IX in mice”, Nature Genet 1993, 5:397-402) and which also contains the human F IX gene and was transfected into the same inbred mouse strain, C57B1/6, as in the preceding experiments, are also depicted in FIG. 1 (broken line, open circles). A very similar short-term expression was obtained with the vector Ad5AE3 F IX, which is the completely E3-deleted counterpart to the first-described vector containing the E3 region. The expression had fallen below the therapeutic threshold value after only about 6 weeks (FIG. 1, unbroken line with diamond symbols).

This direct comparison of an E3-positive F IX vector with an E3-negative F IX vector demonstrates the trans-gene-stabilizing effect of E3:

Another important and novel result of these in-vivo long-term experiments was the observation that the Ad5E3⁺F IX vector exhibited highly significantly improved long-term stability in recipients which were given transient anti-CD4 treatment. The factor IX plasma levels in the anti-CD4 group were still four times over the therapeutic threshold of 5% even six months after the gene transfer whereas the control group had at this time already declined to sub therapeutic levels.

1. An adenoviral vector for gene transfer, which comprises the partial or complete E3 region of wild-type adenovirus type 5 and the gene which is to be transferred and which possesses therapeutic potential.

2. An adenoviral vector, Ad5E3⁺ΔE1, as claimed in claim 1.

3. An adenoviral as claimed in claim 1, into which the gene for human coagulation factor IX is integrated.

4. An adenoviral vector as claim ed in claim 1, into which the gene for human endothelin 1 is integrated.

5. An adenoviral vector as claimed in claim 1, into which the gene for α1-antitrypsin is integrated.

6. An adenoviral vector as claimed in claim 1, into which the gene for human coagulation factor VIII or a functional part thereof is integrated.

7. A combination preparation, which comprises an adenoviral vector as claimed in claim 1 and a means for the anti-CD4 treatment.

8. A combination preparation as claimed in claim 7, wherein the mean for the anti-CD4 treatment brings about receptor blockade or T cell depletion.
